(19) United States
(12) Patent Application Publication
(10) Pub. No.: US 2018/0126043 A1
(43) Pub. Date: May 10, 2018

(54) Sheath Introducer For Peripheral Artery Catheterization Procedures

(71) Applicant: Mladen I. Vidovich, Chicago, IL (US)

(72) Inventor: Mladen I. Vidovich, Chicago, IL (US)

(21) Appl. No.: 15/729,614

(22) Filed: Oct. 10, 2017

Related U.S. Application Data
(60) Provisional application No. 62/406,644, filed on Oct. 11, 2016.

(51) Int. Cl. A61L 29/16; A61M 25/06; A61M 25/09; A61L 29/08; A61L 29/14; A61L 33/00

(52) U.S. Cl. CPC A61L 29/16; A61M 25/065; A61M 25/09041; A61M 25/0662; A61L 29/08; A61L 29/14; A61L 2300/416; A61M 2205/0238; A61M 2205/0205; A61L 2300/45; A61L 2300/42; A61L 2300/41; A61L 33/0011

(57) ABSTRACT

A sheath introducer facilitates the insertion of a catheter into a peripheral artery. The sheath introducer comprises longitudinally extending exterior and interior surfaces, the interior surface defining a lumen in which blood flow occurs. The sheath introducer exterior surface has a hydrophilic material at least partially coated thereon and at least one biologically active agent disposed on the sheath introducer exterior surface. The at least one biologically active agent can be selected from the group consisting of an anticoagulant agent, an antiplatelet agent, an antiproliferative agent, an antibiotics agent, an anti-inflammatory agent, and an antivasospasmodic agent. In a preferred embodiment, the at least one biologically active agent comprises first, second and third biologically active agents, where the first biologically active agent is an antiproliferative agent, the second biologically active agent is an anticoagulant, and the third biologically active agent is an antivasospasmodic agent.

### CROSS-REFERENCE TO RELATED APPLICATION

This application is related to and claims priority benefits from U.S. provisional patent application Ser. No. 62/406,644 filed on Oct. 11, 2016, entitled “Coated Device for Insertion into a Peripheral Artery”. The '644 provisional application is hereby incorporated by reference herein in its entirety.

### FIELD OF THE INVENTION

The present invention relates to medical catheterization procedures and, in particular, to a sheath introducer, in which the sheath introducer for insertion into a peripheral artery has a hydrophilic coating and further comprises at least one biologically active agent.

### BACKGROUND OF THE INVENTION

Endovascular peripheral arterial lines are commonly employed for diagnostic, therapeutic and therapeutic monitoring purposes because they provide ready and convenient access to the heart and other areas of the human body, and are generally considered to be safe. Nonetheless, complications associated with endovascular peripheral arterial procedures, including temporary and permanent occlusion, hemorrhage, infection, ischemia, thrombosis, embolization and neuronal or adjacent structure injury, have been reported in the medical literature.

For example, cardiac catheterization is a minimally invasive procedure commonly used to diagnose and treat heart conditions. During catheterization, the catheter is inserted into the circulatory system under x-ray guidance in order to obtain information about blood flow and pressures within the heart and to determine whether there are obstructions within the blood vessels feeding the heart muscle (coronary arteries). Obstructions of the arteries are caused by plaque buildup, and when severe enough they can cause a variety of symptoms including chest pain and shortness of breath. A catheterization may be recommended on an elective basis if the symptoms are stable. A catheterization may also be required on an emergency basis if the symptoms are sudden and the treating physician is concerned that the symptoms could represent an active or impending heart attack. On the basis of the location and number of obstructions, the treatment plan can include the delivery by the catheter of specialized medications and possibly the placement of a stent or referral for bypass surgery to improve blood flow to the heart muscle and alleviate symptoms.

The catheters necessary for cardiac catheterization can be inserted either into the femoral artery (in the groin), the brachial artery (in the upper arm), or into the radial or ulnar artery (both in the wrist). The femoral artery is a larger vessel and provides a more direct route to the heart. Because of these advantages, the femoral artery has become the standard entry site for catheterization procedures. Similarly, for peripheral arterial diagnostic and therapeutic procedures, arteries in the lower leg (for example, pedal arteries) can be used as an entry site.

Any catheter placement into a blood vessel is associated with a risk of bleeding. After removal of the catheter from the femoral artery, the patient needs to lie flat without bending the leg for 2 to 6 hours to allow the artery to heal. In some cases, even with prolonged immobility, internal bleeding can occur and can be severe enough to require blood transfusions or surgery to repair the femoral artery. To shorten the time to ambulation, femoral closure devices, such as Abbott Vascular's Proglide and Terumo Interventional Systems' Angio-Seal closure devices.

Although the complications with catheterizations using a femoral artery entry site are infrequent, there has been a recent increase, however, in the use of the radial artery as an entry site for cardiac catheterization procedures. Complications have been shown to be less common and less severe when the catheter is inserted in the wrist via the radial or ulnar arteries. Because the radial artery is smaller and located closer to the skin surface, internal bleeding is diminished, or in some cases eliminated, and external bleeding can be managed via external compression. After the catheter is removed from the radial artery, a compression device is placed around the wrist to apply pressure to the artery, thereby reducing the need for the patient to remain immobile. Patients generally find radial catheterization more comfortable than femoral catheterization because patients are able to sit up, walk, and eat more quickly than with femoral catheterization. This is a particular advantage for patients with back problems who must avoid placement of pressure on the leg and prolonged immobility.

Although this application focuses particularly on radial artery entry sites, its teachings apply equivalently to other peripheral vessel entry sites, such as ulnar, pedal and other peripheral arteries. In general, these teachings apply to peripheral arteries located either below the knee or below the elbow, with a diameter of approximately 5 mm or less.

In a radial cardiac catheterization procedure, an introducer needle is first inserted into the radial artery to facilitate the insertion of a guidewire into the radial artery. The needle is then removed, leaving the guidewire in place within the artery. A tubular dilator with a tapered distal end is then threaded around the guidewire, together with a coaxial tubular sheath introducer that surrounds the dilator. The dilator can then be removed, along with the guidewire, leaving the sheath introducer by itself within the radial artery. The sheath introducer then becomes the conduit through which a catheter can be inserted and guided to the heart or other location to be treated, and the coronary angiogram and/or angioplasty and/or stent placement can be performed. Once the catheterization procedure is complete, the catheter and sheath introducer are withdrawn from the radial artery, and a compression device is placed on the wrist. The patient is typically allowed to sit up and eat shortly after the procedure, but it is recommended that no undue stress be put on the radial artery as it heals. By the third day after the procedure, normal activity with the hand can generally be resumed. See Nicholas R. Balaji, et al., Circulation. 2011, 124: e407-e408.

Once the sheath introducer is installed, and before insertion of the catheter into the peripheral artery, medications (sometimes referred to collectively as a “radial artery cocktail”) are typically introduced through the sheath introducer to relax the radial artery. A blood thinner is often introduced to discourage the formation of clots in or near the artery entry site. Antivasospasmodic and other vasoactive drugs can also be administered through the sheath introducer (for example, calcium channel blockers such as Verapamil) to inhibit vasospasms, which can result in the narrowing of the blood vessel, and restriction of blood flow, and can also impede passage of the catheter through the blood vessel, and removal of the catheter and sheath introducer from the blood vessel.

In about 5% of patient cases, permanent obstruction or closure of the radial (or ulnar) artery can occur. This is believed to be due to vessel injury resulting from the sheath introducer insertion and resulting clot formation (thrombosis). While radial artery occlusion is frequently without consequences (asymptomatic), in rare instances this can result in loss of function of the body parts affected by loss of blood flow.

For patients that require frequent insertion of catheter sheath introducers, repeated introduction of catheters and similar endovascular medical devices can cause irreversible vessel thickening that effectively limits the number of sites available for insertion. In extreme cases, where all available insertion sites are used up, further treatment via catheterization can be precluded. The radial artery can also decrease in diameter over time, also making unavailable further catheter procedures.

Finally, there is also a risk during the insertion and/or use and/or removal of a catheter sheath introducer into an artery, that a blood clot could form and cause a thrombosis to travel away from the entry site to a distal site such as the lungs or brain. Accordingly, there is an ongoing need for catheter sheath introducers and similar endovascular devices that mediate the risks associated with insertion of the sheath introducers into narrower peripheral arteries, particularly the radial artery.

### SUMMARY OF THE INVENTION

Shortcomings of conventional peripheral artery sheath introducers are overcome by an improved sheath introducer for insertion of a catheter into a peripheral artery. With the present improved sheath introducer, a formerly passive device for introducing a catheter into a blood vessel is made active by including functionality, particularly in the form of biologically active agents, that mediates at least some of the negative physical effects caused by injury to a blood vessel at the catheter entry site.

The present sheath introducer comprises longitudinally extending exterior and interior surfaces, with the interior surface defining a lumen in the center of the sheath introducer in which blood flow occurs. The sheath introducer exterior surface has a hydrophilic material at least partially coated thereon. At least one biologically active agent is disposed on the sheath introducer exterior surface. In some embodiments, the biologically active agent can be dispersed or embedded in the hydrophilic coating.

In one embodiment, the sheath introducer has an outside diameter of less than 5 mm. The at least one biologically active agent can be bonded to or embedded in the hydrophilic coating layer. The at least one biologically active agent can be one that is released in a controlled fashion from the hydrophilic coating layer.

In another embodiment of the present sheath introducer, the at least one biologically active agent comprises first and second biologically active agents. The first and second biologically active agents can be disposed coaxially from the exterior surface of the sheath introducer, such that the first and second biologically active agents are disposed in strips extending in the longitudinal direction of the sheath introducer.

In another embodiment, the first and second biologically active agents are disposed on the exterior surface of the sheath introducer such that the first and second biologically active agents are arranged in patches in the longitudinal direction of the sheath introducer. The first and second biologically active agents can also be disposed on the exterior surface in a series of discrete rings arranged in the longitudinal direction of the sheath introducer. In another embodiment, the biologically active agents can be disposed on the exterior surface in a spiral arrangement, similar to a barber pole.

In embodiments of the present sheath introducer, the at least one biologically active agent is selected from the group consisting of an anticoagulant agent, an antiplatelet agent, an antiproliferative agent, an antibiotics agent, an anti-inflammatory agent, and an antivasospasmodic agent.

An arterial catheterization kit comprises a sheath introducer for insertion of a catheter into a peripheral artery. The sheath introducer comprises longitudinally extending exterior and interior surfaces. The interior surface defines a cavity in the center of the sheath introducer. The sheath introducer exterior surface has a hydrophilic material at least partially coated thereon. The at least one biologically active agent is disposed on the sheath introducer exterior surface.

In a preferred embodiment, the sheath introducer has an outside diameter of 5 mm or less.

### BRIEF DESCRIPTION OF THE DRAWINGS

FIG. 1 is a simplified diagram of the vascular anatomy of a human hand.

FIG. 2 is a schematic diagram of a radial artery catheterization assembly that includes the present sheath introducer.

FIG. 3 is a top view of a guidewire introducer needle.

FIGS. 4A and 4B are schematic illustrations showing end and perspective views, respectively, of a prior art sheath introducer for arterial catheterization procedures in which the sheath introducer has a hydrophilic material coating applied to its exterior surface.

FIGS. 5A and 5B are schematic illustrations showing end and perspective views, respectively, of an embodiment of the present sheath introducer, in which first and second biologically active agents are disposed coaxially and extend from the exterior surface of the sheath introducer having a hydrophilic material coating applied to its exterior surface.

FIGS. 6A and 6B are schematic illustrations showing end and perspective views, respectively, of an embodiment of the present sheath introducer, in which one or more biologically active agents are dispersed or embedded in the hydrophilic material that is applied as a coating to the exterior surface of the sheath introducer.

FIGS. 7A and 7B are schematic illustrations showing end and perspective views, respectively, of an embodiment of the present sheath introducer, in which a biologically active agent is disposed in strips extending in the longitudinal direction on the exterior surface of the sheath introducer having a hydrophilic material coating applied to its exterior surface.

FIGS. 8A and 8B are schematic illustrations showing end and perspective views, respectively, of an embodiment of the present sheath introducer, in which a biologically active agent is arranged in discrete rings arranged in the longitudinal direction of the sheath introducer having a hydrophilic material coating applied to its exterior surface.

FIGS. 9A and 9B are schematic illustrations showing end and perspective views, respectively, of an embodiment of the present sheath introducer, in which first and second biologically active agents are disposed in alternating strips extending in the longitudinal direction on the exterior surface of the sheath introducer having a hydrophilic material coating applied to its exterior surface.

FIGS. 10A and 10B are schematic illustrations showing end and perspective views, respectively, of an embodiment of the present sheath introducer, in which first and second biologically active agents are disposed in alternating discrete rings arranged in the longitudinal direction on the exterior surface of the sheath introducer having a hydrophilic material coating applied to its exterior surface.

FIGS. 11A and 11B are schematic illustrations showing end and perspective views, respectively, of an embodiment of the present sheath introducer, in which first and second biologically active agents are disposed in strips containing alternating patches of each of the first and second biologically active agents and extend in the longitudinal direction on the exterior surface of the sheath introducer having a hydrophilic material coating applied to its exterior surface.

FIGS. 12A and 12B are schematic illustrations showing end and perspective views, respectively, of another embodiment of the present sheath introducer, in which first and second biologically active agents are disposed coaxially and extend from the exterior surface of the sheath introducer having a hydrophilic material coating applied to its exterior surface.

FIGS. 13A and 13B are schematic illustrations showing end and perspective views, respectively, of another embodiment of the present sheath introducer, in which first, second and third biologically active agents are disposed coaxially and extend from the exterior surface of the sheath introducer having a hydrophilic material coating applied to its exterior surface.

### DETAILED DESCRIPTION OF ILLUSTRATIVE EMBODIMENTS

As used herein, “arterial line” refers to a thin catheter inserted into an artery having a diameter of less than about 5 mm. In some embodiments, the arterial line has a diameter of about 2 mm to about 5 mm. The arterial line is typically inserted in the wrist (radial artery); but can also be inserted into the elbow (brachial artery), groin (femoral artery), foot (pedal; dorsalis pedis artery) or the inside of the wrist (ulnar artery) although an artery that is not an end-artery can theoretically be employed. In general, when selecting an artery for use as an entry site for a catheter, it is highly recommended that there is collateral circulation to the area affected by the chosen artery so that peripheral circulation is maintained by another artery even if circulation is disturbed in the cannulated artery.

“Catheter sheath” or “sheath” or “sheath introducer”, as used herein, means a device which is used to introduce medical devices into a human peripheral artery which comprise a hollow tube, typically made of a flexible material through which other medical devices are introduced into the vascular system. The proximal end of the sheath is exterior to the body, while the majority of the length of the sheath is within the lumen of a vessel of the vascular system. The sheath introducer typically has a length of about 10 cm to about 20 cm and a diameter sufficient to allow passage of the device through the sheath and into the artery. In certain embodiments, the sheath has a diameter of about 5 French to about 7 French. French are units which correspond to the internal diameter of the sheath, and the external diameter of subsequent medical devices inserted into sheath, such as catheters. One French is one-third of a millimeter. Although wall thicknesses of the sheath can vary, it is preferable to have as thin a wall as possible, while retaining the structural robustness of the sheath.

The present sheath introducer comprises a hydrophilic coating to reduce friction as the device is inserted into the radial artery of a patient. Suitable friction lowering coatings are disclosed, for example, in U.S. Pat. Nos. 4,119,094; 4,100,309; 4,500,676; 4,487,865; 4,642,267; 4,769,013; 5,041,100; 6,926,919; 7,297,174; and International Patent Publication Nos. WO2003/061631A1 and WO2001/039811A1.

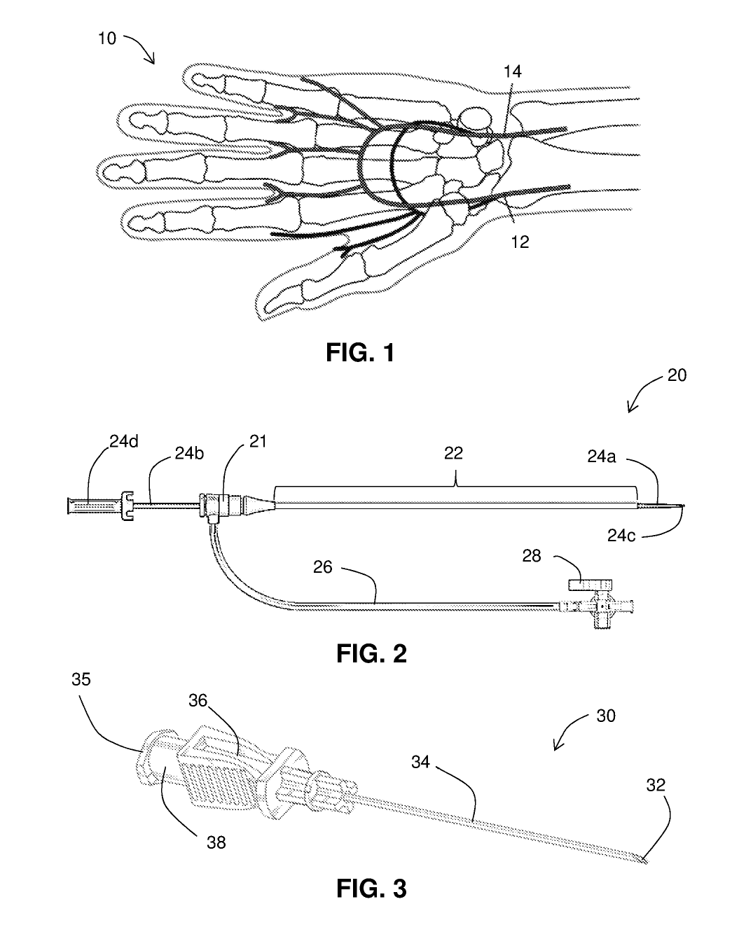

Turning to the figures, FIG. 1 is a simplified diagram of the vascular anatomy of a human hand 10, in which the location of radial artery 12 and ulnar artery 14 are shown.

FIG. 2 schematically illustrates a radial artery catheterization assembly 20. Assembly 20 includes a tubular sheath introducer 22 with a tubular dilator extending longitudinally through sheath introducer 22. The tubular dilator includes a distal portion 24a ending in a tapered tip 24c and a proximal portion 24b with an end cap 24d. A side tube 16 extends from hemostatic valve 21. Fluids, including medications and other biologically active agents, can be introduced to sheath introducer 22 via a three-way stopcock 28.

As shown in FIG. 3, a guidewire introducer needle 30 includes a hollow shaft 34 with a beveled sharpened distal end 32. A gripping portion 36 is located on the proximal end of needle shaft 34. A catheter needle end cap 38, located on the proximal side of gripping portion 26, has an annular port 35 through which a guidewire (not shown) can be threaded toward and through beveled sharpened distal end 32 and into the blood vessel into which needle 30 has been inserted.

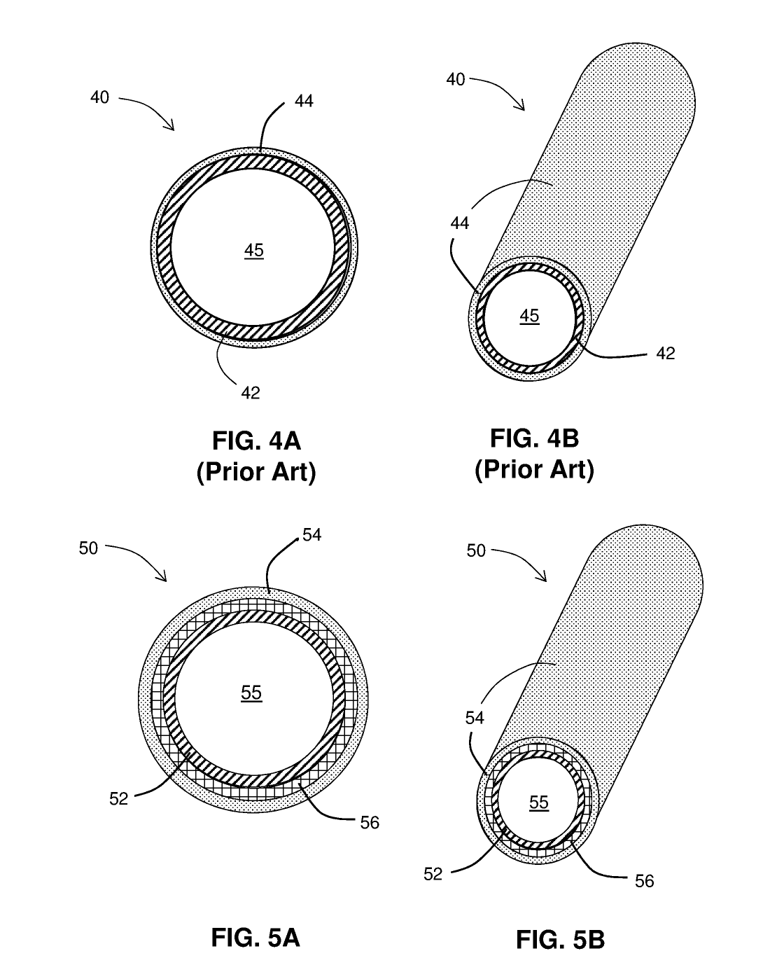

FIGS. 4A and 4B illustrate a prior art sheath introducer 40 for arterial catheterization procedures. Sheath introducer 40 has a sheath body 42, the interior surface of which forms a central lumen 45 through which blood flow occurs. A coating of hydrophilic material 44 is applied to exterior surface of sheath introducer 40.

FIGS. 5A and 5B illustrate a sheath introducer 50 having a sheath body 52, the interior surface of which forms a central lumen 55 through which blood flow occurs. A biologically active agent 56 is disposed on the exterior surface of sheath introducer 50 having a hydrophilic material coating applied to its exterior surface. A coating of hydrophilic material 54 is also applied to exterior surface of sheath introducer 50.

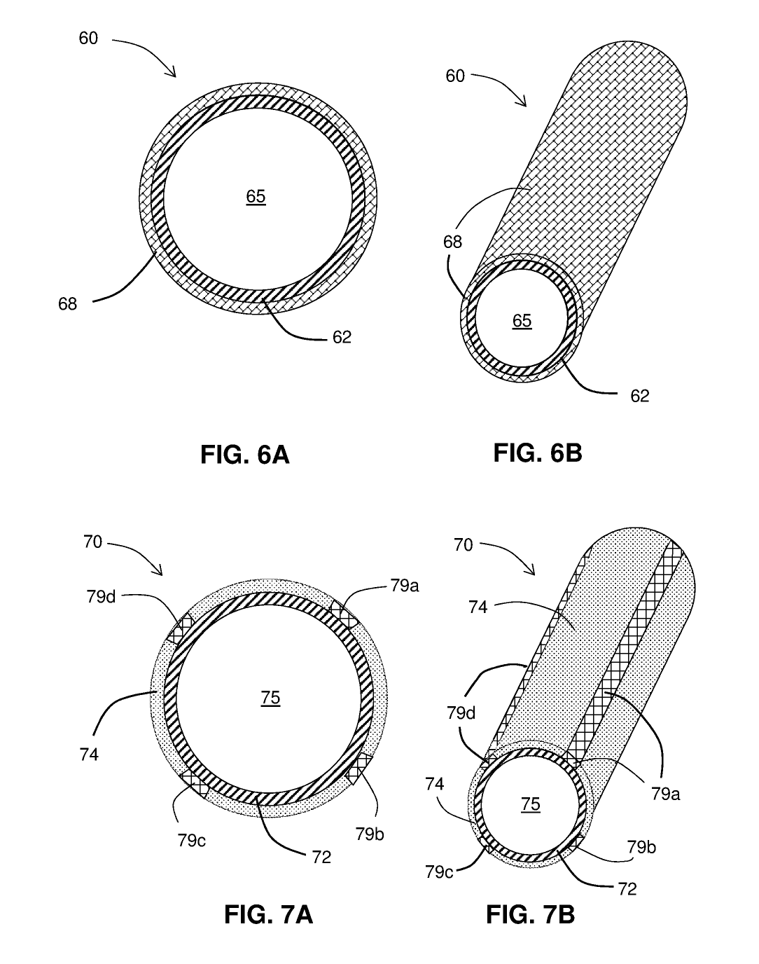

FIGS. 6A and 6B illustrate a sheath introducer 60 having a sheath body 62, the interior surface of which forms a central lumen 65 through which blood flow occurs. One or more biologically active agents are dispersed or embedded in the hydrophilic material that is applied as a coating 68 to the exterior surface of sheath introducer 60.

FIGS. 7A and 7B illustrate a sheath introducer 70 having a sheath body 72, the interior surface of which forms a central lumen 75 through which blood flow occurs. A biologically active agent is disposed in strips 79a, 79b, 79c and 79d extending in the longitudinal direction on the exterior surface of sheath introducer 70. A coating of hydrophilic material 74 is also applied to exterior surface of sheath introducer 70.

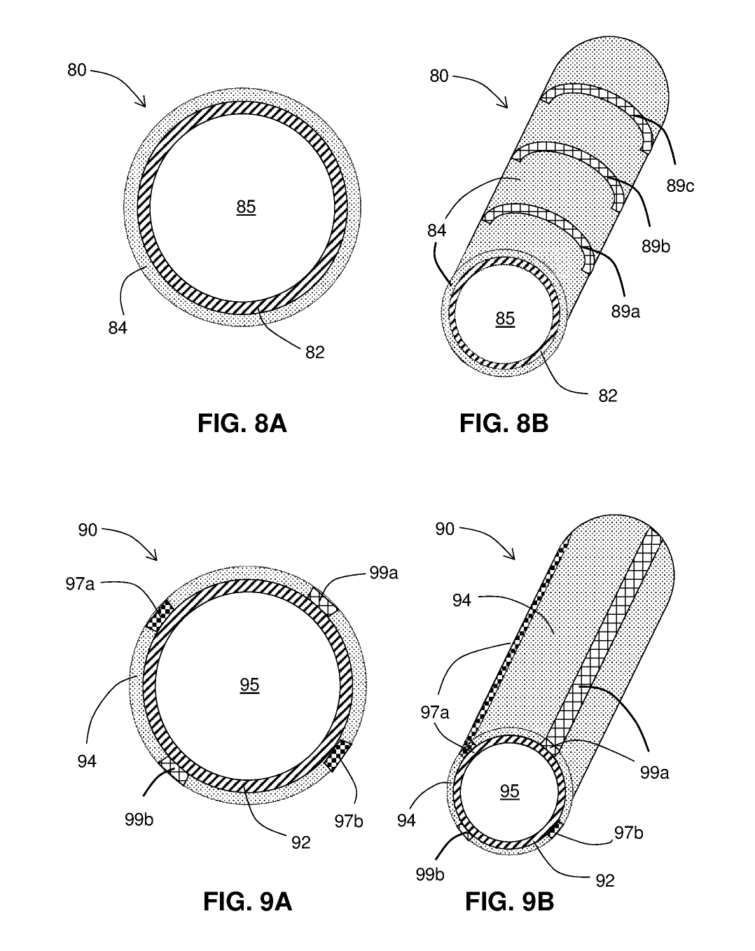

FIGS. 8A and 8B illustrate a sheath introducer 80 having a sheath body 82, the interior surface of which forms a central lumen 85 through which blood flow occurs. A biologically active agent is arranged in discrete rings 89a, 89b and 89c arranged in the longitudinal direction of sheath introducer 80. A coating of hydrophilic material 84 is also applied to exterior surface of sheath introducer 80.

FIGS. 9A and 9B illustrate a sheath introducer 90 having a sheath body 92, the interior surface of which forms a central lumen 95 through which blood flow occurs. A first biologically active agent is disposed in strips 99a and 99b extending in the longitudinal direction on the exterior surface of sheath introducer 90. A second biologically active agent is disposed in strips 97a and 97b extending in the longitudinal direction on the exterior surface of sheath introducer 90. In the circumferential direction, first biologically active agent strips 99a and 99b are arranged alternatingly with second biologically active agent strips 97a and 97b A coating of hydrophilic material 94 is also applied to exterior surface of sheath introducer 90.

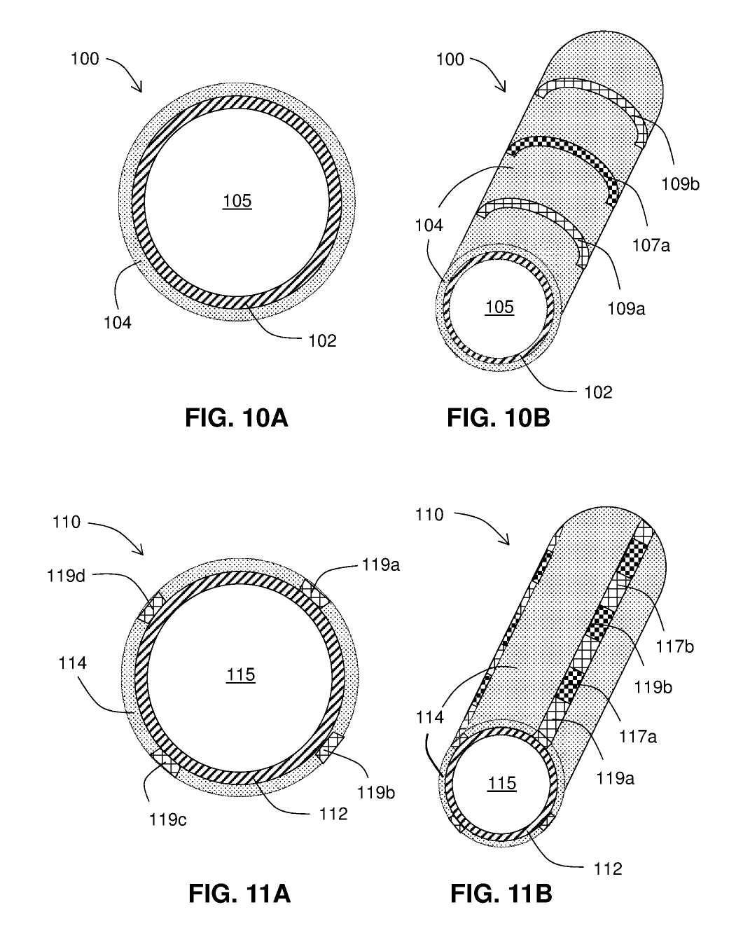

FIGS. 10A and 10B illustrate a sheath introducer 100 having a sheath body 102, the interior surface of which forms a central lumen 105 through which blood flow occurs. A first biologically active agent is disposed in discrete rings 109a and 109b arranged in the longitudinal direction on the exterior surface of sheath introducer 100. A second biologically active agent is disposed in a discrete ring 107a exterior surface of sheath introducer 100 and between first biologically active agent rings 109a and 109b. A coating of hydrophilic material 104 is applied to exterior surface of sheath introducer 100.

FIGS. 11A and 11B illustrate a sheath introducer 110 having a sheath body 112, the interior surface of which forms a central lumen 115 through which blood flow occurs. A first biologically active agent is disposed in patches 119a and 119b arranged in strips extending in the longitudinal direction on the exterior surface of sheath introducer 110. A second biologically active agent is disposed in patches 117a and 117b arranged in strips extending in the longitudinal direction on the exterior surface of sheath introducer 110. First biologically active agent patches 119a and 119b are arranged in an alternating configuration with second biologically active agent patches 117a and 117b. A coating of hydrophilic material 114 is applied to exterior surface of sheath introducer 110.

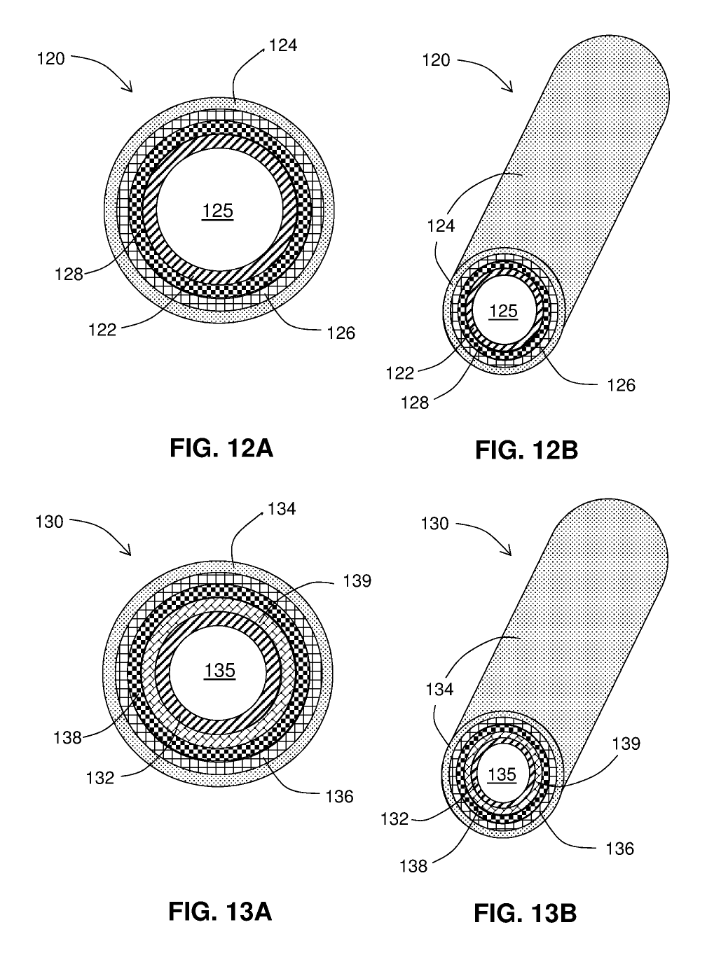

FIGS. 12A and 12B illustrate a sheath introducer 120 having a sheath body 122, the interior surface of which forms a central lumen 125 through which blood flow occurs. A first biologically active agent 128 is disposed coaxially from the exterior surface of sheath introducer 120. A second biologically active agent 126 is disposed coaxially with respect to first biologically active agent 128. A coating of hydrophilic material 124 is applied to exterior surface of sheath introducer 120.

FIGS. 13A and 13B illustrate a sheath introducer 130 having a sheath body 132, the interior surface of which forms a central lumen 135 through which blood flow occurs. A first biologically active agent 139 is disposed coaxially from the exterior surface of sheath introducer 130. A second biologically active agent 138 is disposed coaxially with respect to first biologically active agent 139. A third biologically active agent 136 is disposed coaxially with respect to first biologically active agent 139 and second biologically active agent 138. A coating of hydrophilic material 134 is applied to exterior surface of sheath introducer 130.

Representative hydrophilic coating materials can include poly(alkylene glycols) including poly(ethylene glycol), poly(ethylene oxide), poly(propylene glycol), poly(ethylene oxide-co-propylene oxide), poly(trimethylene glycol), poly(tetramethylene glycol), and mixtures thereof, N-vinylpyrrolidinone, hydroxyethylmethacrylate, and N-dimethylaminoethylmethacrylate.

The hydrophilic coating is applied to the sheath in such manner that the material provides an adherent matrix, suitable for the incorporation of drugs on the sheath. The polymer coating can be applied to the surface of the sheath in one of the final steps of its manufacturing, but prior to sterilization. The polymeric coating can be applied as a solution in a volatile organic solvent, via a spray process, a dip-coating process, or otherwise. This can be followed by a treatment of the coated product at elevated temperature and/or vacuum, in order to facilitate evaporation of residual solvent molecules, and/or in order to achieve firm attachment of the polymer coating to the surface. The copolymers as described above can be dissolved in a volatile organic solvent, and can be applied to the product via a dip-coating procedure or via a spray process. Other processes resulting in a suitable coating on the metal can also be used.

An effective amount of the biologically active agents can be incorporated into the hydrophilic coating layer. As used herein, “effective amount” means an amount of pharmacologically active agent that is nontoxic but sufficient to provide the desired local or systemic effect and performance at a reasonable benefit/risk ratio attending any medical treatment.

Once the sheath introducer is placed in the artery, the hydrophilic coating absorbs the body fluids and the biologically active agents elute from their location in the sheath introducer at predetermined rates for a specific period of time during the medical procedure.

As used herein, “biologically active agent” means a drug or other substance that has therapeutic value to a living organism including without limitation anticoagulants, antiplatelet agents, antiproliferative agents, antibiotic agents, anti-inflammatory agents, and mixtures thereof.

“Anticoagulant” refers to a class of drugs that work to prevent the coagulation (clotting) of blood. Anticoagulants include antithrombics, fibrinolytics, and thrombolytics. Representative anticoagulants include, but are not limited to, antithrombotics such as heparin and low molecular weight heparin, factor Xa inhibitors such as fondaparinux (Arixtra) and idraparinux, direct thrombin inhibitors such as bivalirudin and argatroban. In one embodiment, the anticoagulant is heparin. Representative anti-inflammatory drugs include classic non-steroidal anti-inflammatory drugs (NSAIDS), such as aspirin, diclofenac, indomethacin, sulindac, ketoprofen, flurbiprofen, ibuprofen, naproxen, piroxicam, tenoxicam, tolmetin, ketorolac, oxaprosin, mefenamic acid, fenoprofen, nambumetone (relafen), acetaminophen (Tylenol®), and mixtures thereof; COX-2 inhibitors, such as nimesulide, NS-398, flosulid, L-745337, celecoxib, rofecoxib, SC-57666, DuP-697, parecoxib sodium, JTE-522, valdecoxib, SC-58125, etoricoxib, RS-57067, L-748780, L-761066, APHS, etodolac, meloxicam, S-2474, and mixtures thereof; glucocorticoids, such as hydrocortisone, cortisone, prednisone, prednisolone, methylprednisolone, meprednisone, triamcinolone, paramethasone, fluprednisolone, betamethasone, dexamethasone, fludrocortisone, desoxycorticosterone, and mixtures thereof; and mixtures thereof.

Anti-proliferative agents inhibit or prevent thickening of blood vessels, particularly peripheral arteries, over time. Anti-proliferative agents are especially effective to mediate injuries to an artery from use as an entry site in multiple catheterization procedures. Representative anti-proliferative agents can include Sirolimus, Everolimus, Zotarolimus, Biolimus A9, and Paclitaxel.

“Antimicrobial agents” and “antimicrobials” as used herein mean an agent which by itself or through assisting the body (immune system) helps the body destroy or resist microorganisms which can be pathogenic (disease causing). The term “antimicrobial agent” includes antibiotics, quorum sensing blockers, surfactants, metal ions, antimicrobial proteins and peptides, antimicrobial polysaccharides, antiseptics, disinfectants, anti-virals, anti-fungals, and combinations thereof.

In an embodiment, the biologically active agents are incorporated into the hydrophilic coating.

In an embodiment, the biologically active agents are bonded to the hydrophilic coating layer or the second coating layer such that they remain proximate to and exert their effect at the site of insertion of the device.

In another embodiment, the biologically active agents are released in a controlled fashion from the hydrophilic coating layer into the general circulation.

In another embodiment, the biologically active agents can be incorporated into voids in the hydrophilic coating layer. The biologically active agents are later eluted from the hydrophilic coating to the body fluids such as blood once the hydrophilic coating is contacted by body fluids.

In an embodiment, the hydrophilic coating comprises one or more anticoagulants. The elution of anticoagulants such as sodium heparin is important to minimizing blood clotting complications during vascular catheterization procedures. In contrast to systemic injections of heparin, the elution of antithombolitic agents from the surface of the coated sheath provides the target delivery or release of the drug at the surface of the invasive material. Therefore, a more direct and effective antithrombolitic treatment is administered.

In another embodiment, the hydrophilic coating can be loaded with one or more antibiotic agents. In this configuration, the catheter sheath could elute the antibiotic directly to the skin-tissue entry point (proximal segment) in order to prevent infections. The puncture site where the catheter enters the skin can be vulnerable to bacterial infection.

In yet another embodiment, different biologically active agents are selectively loaded to different portions of hydrophilic coating. For example, the device could be coated with a hydrophilic coating eluting antithrombogenic drug in blood contacting areas and an antibiotic drug eluting in other areas where the device comes in contact with tissue, such as the entry point where the medical device penetrates the skin-tissue.

In another embodiment, a second coating layer comprising one or more biologically active agents can be applied over the hydrophilic coating. The biologically active agents can be incorporated into or conjugated to the second coating layer. It is preferred that the coating substantially cover the entire sheath introducer surface, but the coating can cover only a portion of the sheath. As described above, the biologically active agents can be bonded to the second coating layer such that they remain proximate to and exert their effect at the site of insertion of the device or released in a controlled fashion from the hydrophilic coating layer into the general circulation.

For example, a second coating layer eluting an antibiotic can be applied to the proximal segment of the sheath and/or a second layer eluting one or more coagulants and/or anti-proliferative agents can be applied in blood-contact areas as described above.

In an embodiment, second coating layer comprises a polymeric film loaded with the biologically active agent(s). Illustrative polymers that can be used for making the polymeric film include polyurethanes, polyethylene terephthalate (PET), PLLA-poly-glycolic acid (PGA) copolymer (PLGA), polycaprolactone (PCL) poly-(hydroxybutyrate/hydroxyvalerate) copolymer (PHBV), poly(vinylpyrrolidone) (PVP), polytetrafluoroethylene (PTFE; tradename Teflon), poly(2-hydroxyethylmethacrylate) (poly-HEMA), poly(etherurethane urea), silicones, acrylics, epoxides, polyesters, urethanes, parlenes, polyphosphazene polymers, fluoropolymers, polyamides, polyolefins, and mixtures thereof. The second layer of the hydrophobic heparinized polymer also has the effect of preventing a burst release of any biologically active agent dispersed in the first layer, resulting in a relatively longer release period of the biologically active agent. It should also be understood that the both the first and second coating layers can contain more than one biologically active agent.

The second coating layer can be applied as described above for the hydrophilic coating layer, for example by applying as a solution in a volatile organic solvent, via a spray process, a dip-coating process, or otherwise.

In yet another embodiment, the hydrophilic coating and/or the second coating layer can be loaded with a biocompatible dye in order to provide a color to the coating. This feature helps in visually inspecting the coating coverage during and after the coating process. Further, an ultraviolet (UV) tracing dye could be added the polymer matrix to render the dye visible only when a UV source is used to illuminate or reveal the coating.

In an embodiment, the biologically active agent comprises an anticoagulant, preferably heparin.

In another embodiment, the biologically active agent comprises one or more antithrombogenic heparinized polymers. Antithrombogenic heparinized polymers are soluble only in organic solvents and are insoluble in water. Antithrombogenic heparin polymers are produced by binding heparin to macromolecules and hydrophobic materials.

Once the hydrophobic heparinized polymer has been prepared, the heparinized polymer layer can be applied directly over the first layer using the solvent evaporation method or other appropriate method. The hydrophobic heparinized polymer is readied for application by combining it with a solvent, such as cyclohexane, thereby forming an aqueous solution having the hydrophobic heparinized polymer suspended therein. The antithrombogenic heparinized polymer and solvent solution can then be applied to the sheath using a dipping process. The solvent is evaporated from the sheath during a drying process; leaving a thin film of the hydrophobic heparinized polymer over the first layer.

The antithrombogenic heparinized polymer layer inhibits coagulation at the implant site. In addition, the second layer inhibits or prevents a burst release of a biologically active agent from the first layer. The second layer also serves to extend the release period of the biologically active agent from the first layer, thereby lengthening the treatment time.

In another embodiment, the coated device is configured have the biologically active agent(s) disposed within the hydrophilic coating layer, the second coating layer or a combination of the hydrophilic coating layer and second polymer layer in a manner that controls the elution of the drug so as to preferentially deliver the drug into arterial tissue adjacent to the device while maintaining a sufficiently low systemic concentration of the drug.

The polymeric coatings and biologically active agent(s) are configured to cooperate so as to form a diffusion pathway (for example, lipophilic, hydrophilic and/or amphipathic) with tissue when the device is disposed in the arterial lumen such that the drug preferentially diffuses into the tissue over a body fluid passing through the lumen so as to retain a sufficiently low concentration in order to inhibit negative systemic side effects.

Examples of suitable biocompatible polymeric materials include a suitable hydrogel, hydrophilic polymer, hydrophobic polymer biodegradable polymers, bioabsorbable polymers, and monomers thereof. Additionally, the coating can include hydrophilic and/or hydrophobic compounds, polypeptides, proteins, amino acids, polyethylene glycols, parylene, heparin, and phosphorylcholine.

While particular elements, embodiments and applications of the present invention have been shown and described, it will be understood, that the invention is not limited thereto since modifications can be made by those skilled in the art without departing from the scope of the present disclosure, particularly in light of the foregoing teachings.

What is claimed is:

1. A sheath introducer for insertion of a catheter into a peripheral artery, the sheath introducer comprising longitudinally extending exterior and interior surfaces, the interior surface defining a lumen in the center of the sheath introducer, the sheath introducer exterior surface having a hydrophilic material at least partially coated thereon and at least one biologically active agent disposed on said exterior surface.

2. The sheath introducer of claim 1, wherein said sheath introducer has an outside diameter of less than 5 mm.

3. The sheath introducer of claim 1, wherein said at least one biologically active agent is bonded to said hydrophilic coating layer.

4. The sheath introducer of claim 1, wherein said at least one biologically active agent is dispersed in said hydrophilic coating layer.

5. The sheath introducer of claim 1, wherein said at least one biologically active agent is embedded in said hydrophilic coating layer.

6. The sheath introducer of claim 1, wherein said at least one biologically active agent is released in a controlled fashion from said hydrophilic coating layer.

7. The sheath introducer of claim 1, wherein said at least one biologically active agent comprises first and second biologically active agents.

8. The sheath introducer of claim 7, wherein said first and second biologically active agents are disposed coaxially on said sheath introducer exterior surface.

9. The sheath introducer of claim 7, wherein said first and second biologically active agents are disposed on said sheath introducer exterior surface, and wherein said first and second biologically active agents are disposed coextensively in the longitudinal direction of said sheath introducer.

10. The sheath introducer of claim 7, wherein said first and second biologically active agents are disposed on said sheath introducer exterior surface, and wherein said first and second biologically active agents are arranged in patches in the longitudinal direction of said sheath introducer.

11. The sheath introducer of claim 7, wherein said first and second biologically active agents are disposed on said sheath introducer exterior surface in a series of discrete rings arranged in the longitudinal direction of said sheath introduce.

12. The sheath introducer of claim 7, wherein said first and second biologically active agents are disposed on said sheath introducer exterior surface in a spiral arrangement in the longitudinal direction of said sheath introducer.

13. The sheath introducer of claim 1, wherein said at least one biologically active agent comprises first, second and third biologically active agents.

14. The sheath introducer of claim 1, wherein said at least one biologically active agent is selected from the group consisting of an anticoagulant agent, an antiplatelet agent, an antiproliferative agent, an antibiotics agent, an anti-inflammatory agent, and an antivasospasmodic agent.

15. The sheath introducer of claim 14, wherein said anticoagulant agent is selected from the group consisting of an antithrombic agent, a fibrinolytic agent, and a thrombolytic agent.

16. The sheath introducer of claim 15, wherein said anticoagulant agent is heparin.

17. The sheath introducer of claim 15, wherein said anticoagulant agent is a factor Xa inhibitor.

18. The sheath introducer of claim 17, wherein said factor Xa inhibitor is one of fondaparinux or idraparinux.

19. The sheath introducer of claim 15, wherein said anticoagulant agent is a direct thrombin inhibitor.

20. The sheath introducer of claim 19, wherein said direct thrombin inhibitor is one of bivalirudin or argatroban.

21. The sheath introducer of claim 14, wherein said anti-inflammatory agent is selected from the group consisting of a non-steroidal anti-inflammatory drug (NSAID), a COX-2 inhibitor, a glucocorticoid, and mixtures thereof.

22. The sheath introducer of claim 14, wherein the anti-inflammatory agent is selected from the group consisting of aspirin, diclofenac, indomethacin, sulindac, ketoprofen, flurbiprofen, ibuprofen, naproxen, piroxicam, tenoxicam, tolmetin, ketorolac, oxaprosin, mefenamic acid, fenoprofen, nambumetone, acetaminophen, nimesulide, NS-398, flosulid, L-745337, celecoxib, rofecoxib, SC-57666, DuP-697, parecoxib sodium, JTE-522, valdecoxib, SC-58125, etoricoxib, RS-57067, L-748780, L-761066, APHS, etodolac, meloxicam, S-2474, hydrocortisone, cortisone, prednisone, prednisolone, methylprednisolone, meprednisone, triamcinolone, paramethasone, fluprednisolone, betamethasone, dexamethasone, fludrocortisone, desoxycorticosterone, and mixtures thereof.

23. The sheath introducer of claim 14, wherein said anti-proliferative agent is selected from the group consisting of sirolimus, everolimus, zotarolimus, biolimus A9, Paclitaxel, and mixtures thereof.

24. The sheath introducer of claim 13, wherein said first biologically active agent is an antiproliferative agent, said second biologically active agent is an anticoagulant, and said third biologically active agent is an antivasospasmodic agent.

25. An arterial catheterization kit comprising a sheath introducer for insertion of a catheter into a peripheral artery, the sheath introducer comprising longitudinally extending exterior and interior surfaces, said interior surface defining a cavity in the center of the sheath introducer, said sheath introducer exterior surface having a hydrophilic material at least partially coated thereon, said at least one biologically active agent disposed between the sheath introducer exterior surface and the center cavity.

26. The arterial catheterization kit of claim 25, wherein said sheath introducer has an outside diameter of less than 5 mm.
